SEQUENCE LISTING (12 sequences)

SEQ ID NO: 1 - protein, 107 aa, Artificial Sequence [sequence omitted]

SEQ ID NO: 2 - protein, 116 aa, Artificial Sequence [sequence omitted]

SEQ ID NO: 3 - protein, 214 aa, Artificial Sequence
  Synthetic Sequence
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 446 aa, Artificial Sequence
  Synthetic Sequence
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 6 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 7 - protein, 453 aa, Artificial Sequence
  Synthetic Sequence
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 214 aa, Artificial Sequence
  Synthetic Sequence
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 451 aa, Artificial Sequence
  Synthetic Sequence
EVKLEESGGGLVQPGGSMKLSCVASGFTFSNYWMNWVRQSPEKGLEWVAEITLKSNNYATHYAESVKGRFTISRDDSKSSVYLQMNNLRPEDTGIYYCTRNDYDRAWFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 10 - protein, 220 aa, Artificial Sequence
  Synthetic Sequence
DIVMSQSPSSLAVSVGEKVTLSCKSSQSLLYSSNQKNYLAWYQQEPGQSPKLLIYWASTRESGVPDRFTGSGSGTDFTLTISSVKAEDLAVYYCQQYYNYPWTFGGGSKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - protein, 446 aa, Artificial Sequence
  Synthetic Sequence
EVQLQESGPSLVKPSQTLSLTCSVTGDSFTSDYWNWIRKFPGNRLEYMGYISYRGVTYYNPSLKSRISITRDTSKNQYYLQLNSVTTEDTATYYCARWGTVPLDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 12 - protein, 219 aa, Artificial Sequence
  Synthetic Sequence
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHIPYTFGGGTKLEMKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2020/0062836 A1
(43) Pub. Date: Feb. 27, 2020

(54) THERAPY FOR METATASTIC COLORECTAL CANCER USING ANTI-VEGFR-2 and ANTI-VEGF-D ANTIBODIES

(71) Applicant: ELI LILLY AND COMPANY, Indianapolis, IN (US)

(72) Inventors: Rebecca Rose HOZAK, Greenfield, IN (US); Tara Suzanne UMBERGER, Franklin, IN (US)

(73) Assignee: Eli Lilly and Company, Indianapolis, IN (US)

(21) Appl. No.: 16/346,634

(22) PCT Filed: Nov. 10, 2017
(86) PCT No.: PCT/US2017/061019

§ 371 Date: May 1, 2019

Related U.S. Application Data
(60) Provisional application No. 62/422,904, filed on Nov. 16, 2016.

(51) Int. Cl. C07K 16/22; C07K 16/28; A61P 35/00; G01N 33/574

(52) U.S. Cl. CPC C07K 16/22; G01N 33/57419; A61P 35/00; C07K 16/2863

(57) ABSTRACT

The invention provides for methods, treatments, and uses for anti-human VEGFR-2 antibodies for the treatment of metastatic colorectal cancer in patients having elevated levels of VEGF-D.

The present invention relates to the field of cancer treatment. More specifically, the present invention relates to the use of an anti-VEGFR-2 antibody, preferably ramucirumab, for the treatment of metastatic colorectal cancer (mCRC) in patients having elevated levels of human vascular endothelial growth factor D (VEGF-D). Measuring the level of VEGF-D is expected to be useful in the selection of patients with mCRC for treatment with ramucirumab.

Ramucirumab is currently approved as a single agent or in combination with paclitaxel, for the treatment of advanced gastric or gastro-esophageal junction adenocarcinoma, with disease progression on or after prior fluoropyrimidine- or platinum-containing chemotherapy; in combination with docetaxel, for treatment of metastatic nonsmall cell lung cancer with disease progression on or after platinum-based chemotherapy; in combination with FOLFIRI, for the treatment of metastatic colorectal cancer with disease progression on or after prior therapy with bevacizumab, oxaliplatin, and a fluoropyrimidine.

VEGF-D is a ligand for the human vascular endothelial growth receptor-2 (VEGFR-2). It has been shown that patients with metastatic colorectal cancer and with lower levels of VEGF-D (as determined by IHC) benefit from bevacizumab treatment and that patients with metastatic colorectal cancer and with higher VEGF-D expression receive less benefit from bevacizumab. British Journal of Cancer 113, 37-45 (2015). It has also been shown that circulating levels of VEGF-D may be predictive of bevacizumab treatment benefit in patients with metastatic colorectal cancer and treated with FOLFOX or FOLFIRI plus bevacizumab or cetuximab, with patients having low VEGF-D levels receiving the greatest bevacizumab benefit. J. Clin. Oncol. 34, 2016 (suppl.; abstract 3597). However, multiple markers were tested, and none, including VEGF-D, were reported to be statistically significant after correction for multiple testing. J. Clin. Oncol. 34, 2016 (suppl.; abstract 3597).

It has surprisingly been found that mCRC patients with elevated VEGF-D plasma levels receive a larger survival benefit from the addition of ramucirumab to FOLFIRI, compared to patients with lower VEGF-D levels.

Ramucirumab is a human IgG1 monoclonal antibody directed against human vascular endothelial growth factor receptor 2 (VEGFR-2). Ramucirumab and methods of making and using ramucirumab have been previously disclosed. Ramucirumab is approved by the United States Food and Drug Administration as a single agent or in combination with paclitaxel, for the treatment of advanced gastric or gastro-esophageal junction adenocarcinoma, with disease progression on or after prior fluoropyrimidine- or platinum-containing chemotherapy; in combination with docetaxel, for the treatment of metastatic non-small cell lung cancer with disease progression on or after platinum-based chemotherapy. Patients with epidermal growth factor receptor (EGFR) or anaplastic lymphoma kinase (ALK) genomic tumor aberrations should have disease progression on FDA-approved therapy for these aberrations prior to receiving CYRAMZA®; and in combination with FOLFIRI (irinotecan, folinic acid, and 5-fluorouracil), for the treatment of metastatic colorectal cancer with disease progression on or after prior therapy with bevacizumab, oxaliplatin, and a fluoropyrimidine.

The present invention is derived from a Phase 3 clinical trial of ramucirumab (“A Randomized, Double-Blind, Multicenter Phase 3 Study of Irinotean, Folinic Acid, and 5-Fluorouracil (FOLFIRI) Plus Ramucirumab or Placebo in Patients with Metastatic Colorectal Carcinoma Progressive During or Following First-Line Combination Therapy With Bevacizumab, Oxaliplatin, and a Fluoropyrimidine) (the “Study”).

As used herein, the term “human VEGFR-2” refers to Human Vascular Endothelial Growth Factor Receptor 2 having the amino acid sequence of SEQ ID NO: 5. VEGFR-2 is also known as KDR.

As used herein, the term “human VEGF-D” refers to Human Vascular Endothelial Growth Factor-D having the amino acid sequence of SEQ ID NO: 6.

A non-limiting example of ramucirumab is CYRAMZA® and has the CAS registry number 947687-13-0. Ramucirumab is an anti-human VEGFR-2 antibody comprising two light chains, each of the light chains having the amino acid sequence of SEQ ID NO: 3, and two heavy chains, each of the heavy chains having the amino acid sequence of SEQ ID NO: 4. The light chain variable region of ramucirumab is that given in SEQ ID NO: 1. The heavy chain variable region of ramucirumab is that given in SEQ ID NO: 2.

The anti-human VEGFR-2 antibody selected will have a sufficiently strong binding affinity for human VEGFR-2. For example, the antibody will generally bind VEGFR-2 with a K_{d} value of between about 100 nM and about 1 pM. Antibody affinities may be determined by a surface plasmon resonance based assay (such as the BIAcore assay is described in WO2005/012359); enzyme-linked immunosorbent assay (ELISA); and competition assays (e.g. a radiolabeled antigen binding assay (RIA)), for example. In one embodiment, Kd is measured by a RIA performed with ramucirumab.

Bevacizumab, is an antibody specific for vascular endothelial growth factor (VEGF), and is approved by the United States Food and Drug Administration as a first-line treatment for patients with metastatic colorectal cancer. Bevacizumab is approved by the United States Food and Drug Administration as a second-line treatment of metastatic colorectal cancer for use in combination with intravenous 5-fluorouracil-based chemotherapy. Bevacizumab is also approved by the United States Food and Drug Administration for use in combination with fluoropyrimidine-irinotecan- or fluoropyrimidine-oxaliplatin-based chemotherapy for the treatment of patients with metastatic colorectal cancer (mCRC) whose disease has progressed while on first-line treatment with a bevacizumab-containing regimen.

“Bevacizumab” as used herein includes Avastin® and other antibodies comprising the heavy chain and light chains shown in SEQ ID NO: 7 and SEQ ID NO: 8, respectively, as well as the following non-limiting examples as shown in Table 1. It is to be recognized that “bevacizumab” as used herein can be made using a variety of cell lines and may exhibit some differences, including but not limited to, differences in glycosylation as a result.

TABLE 1
Non-Limiting Examples of
Bevacizumab:

Avastin ®
ABP215
BCD-021
PF-06439535
RPH-001
Non-listed Biosimilars and
interchangeable Biologics of
Bevacizumab

As used herein, the terms “treating,” “treat,” or “treatment” refer to restraining, slowing, lessening, reducing, or reversing the progression or severity of an existing symptom, disorder, condition, or disease, or ameliorating clinical symptoms of a condition. Beneficial or desired clinical results include, but are not limited to, alleviation of symptoms, diminishment of the extent of a disease or disorder, stabilization of a disease or disorder (i.e., where the disease or disorder does not worsen), delay or slowing of the progression of a disease or disorder, amelioration or palliation of the disease or disorder, and remission (whether partial or total) of the disease or disorder, whether detectable or undetectable. Treatment can also mean prolonging survival as compared to expected survival if not receiving treatment. Those in need of treatment include those already with the disease. In one embodiment, the present invention can be used as a medicament.

As used herein, the term “cancer” refers to or describes the physiological condition in mammals that is typically characterized by unregulated cell growth. Included in this definition are benign and malignant cancers.

In the methods of the present invention, a therapeutically effective amount of an antibody of the invention is administered to a mammal or patient in need thereof. Additionally, the pharmaceutical compositions of the invention may include a therapeutically effective amount of ramucirumab of the invention.

A “therapeutically effective amount,” refers to an amount effective, at dosages and for periods of time necessary, to achieve the desired therapeutic result. A therapeutically effective amount can be readily determined by the attending diagnostician, as one skilled in the art, by the use of known techniques and by observing results obtained under analogous circumstances. In determining the therapeutically effective amount for a patient, a number of factors are considered by the attending diagnostician, including, but not limited to: the species of patient; its size, age, and general health; the specific disease or disorder involved; the target site; the degree of the severity of the disease or disorder; the response of the individual patient; the particular compound administered; the mode of administration; the bioavailability characteristics of the preparation administered; the dose regimen selected; the use of concomitant medication; other medications administered; and other relevant circumstances. A therapeutically effective amount is also one in which any toxic or detrimental effects of the antibody or antibody portion are outweighed by the therapeutically beneficial effects.

Generally, dosage regimens may be adjusted to provide the optimum desired response (e.g., a therapeutic response). Treatment dosages may be titrated using routine methods known to those of skill in the art to optimize safety and efficacy. Dosing schedules will typically range from a single bolus dosage or continuous infusion, to multiple administrations per day (e.g., every 4-6 hours), or as indicated by the treating physician and the patient's condition. Dosing frequencies of the antibody will be determined by the physicians treating the patient and may be given daily, three times per week, weekly, every two weeks, or less often, and more preferably every two-weeks. Dosing amounts of the antibody will also be determined by the physicians treating the patient and may fall within customary ranges, more preferably about 8 mg/kg.

In some instances, dosage levels below the lower limit of the aforesaid dosing for ramucirumab may be more than adequate, while in other cases larger doses may be employed with acceptable side effects, and therefore the above dosage amount is not intended to limit the scope of the invention in any way.

The therapeutically effective amount of the treatment of the invention can be measured by various endpoints commonly used in evaluating cancer treatments, including, but not limited to: extending survival (including Overall Survival (OS) and Progression Free Survival (PFS)); resulting in an objective response (including a Complete Response (CR) or a Partial Response (PR)); tumor regression, tumor weight or size shrinkage, longer time to disease progression, increased duration of survival, longer PFS, improved OS rate, increased duration of response, and improved quality of life and/or improving signs or symptoms of cancer.

As used herein, the term “progressive disease” (PD) refers to least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progression.

As used herein, the term “partial response,” (PR) refers to at least a 30% decrease in the sum of diameters of target lesions, taking as reference the baseline sum diameters.

As used herein, the term “complete response” (CR) refers to the disappearance of all non-nodal target lesions with the short axes of any target lymph nodes reduced to <10 mm.

As used herein, the term “stable disease” (SD) refers to neither sufficient shrinkage for PR nor sufficient increase to qualify for PD, taking as reference the smallest sum of diameters while on study.

As used herein, the term “objective response rate” (ORR) is equal to the proportion of patients achieving a best overall response of partial or complete response (PR+CR) according to RECIST 1.1.

As used herein, the term “overall survival” (OS) refers to the percentage of patients remaining alive for a defined period of time, such as 1 year, 5 years, etc. from the time of diagnosis or treatment. In a preferred embodiment, OS refers to the time from the date of randomization in the Study to the date of death from any cause. If the patient is alive at the end of the follow-up period or is lost to follow-up, OS data is censored on the last date the patient is known to be alive. Overall survival is evaluated by the Kaplan-Meier method, and a 95% confidence interval (CI) is provided for the median OS in each treatment arm.

As used herein, the term “progression-free survival” (PFS) refers to the patient remaining alive without the cancer progressing or getting worse. In a preferred aspect of the invention, PFS is defined as the time from randomization in the Study until the first radiographic documentation of objective progression as defined by RECIST (Version 1.1), or death from any cause. Patients who die without a reported prior progression will be considered to have progressed on the day of their death. Patients who did not progress or are lost to follow-up will be censored at the day of their last radiographic tumor assessment. In a preferred embodiment, progression-free survival is analyzed using the log-rank test, stratified by geographic region (North America versus Europe versus all other regions), KRAS status (mutant versus wild-type), and time to disease progression after beginning first-line treatment (<6 months versus ≥6 months).

As used herein, the term “disease control rate” (DCR) refers to lack of disease progression and rate thereof. It refers to the group of patients with a best overall response categorized as CR, PR or SD (specifically excluding the patients with PD), wherein the best overall response is the best response recorded from the start of treatment until PD.

As used herein, the term “clinical benefit rate,” refers to SD or better at 12 weeks. The tumor response rate of SD or better (i.e. CR+PR+SD) at 12 weeks is defined as the proportion of patients with a response of SD or better, as defined by RECIST 1.1, at 12 weeks following the first dose of study therapy. Patients will be considered “failure” if they die or if radiographic evaluation indicates a response of PD at 12 weeks or before.

As used herein, the term “extending survival” or “prolonged survival” which are used interchangeably herein, is meant as increasing OS or PFS in a treated patient relative to i) an untreated patient, ii) a patient treated with less than all of the anti-tumor agents in a particular combination therapy, or iii) a control treatment protocol. Survival is monitored following the initiation of treatment or following the initial diagnosis of cancer.

In the present invention, any suitable method or route can be used to administer ramucirumab of the invention; intravenous (i.v.) administration is the preferred route. It should be emphasized, however, that the present invention is not limited to any particular method or route of administration.

The anti-human VEGFR-2 antibodies, including but not limited to ramucirumab, where used in a patient for the purpose of treatment, is preferably formulated as a pharmaceutical composition. Such pharmaceutical compositions and processes for preparing the same are well known in the art. See, e.g. Remington: The Science and Practice of Pharmacy (Gennaro A., et al., eds., 19th ed., Mack Publishing Co., 1995).

VEGF-D can be collected from patient blood by techniques known in the art. Levels of VEGF—are measured as described herein. Serum and plasma can be derived by methods known in the art. The level of VEGF-D can be predictive of or indicative of treatment efficacy with ramucirumab.

Unless indicated otherwise, the term “antibody” refers to an immunoglobulin molecule comprising two heavy chains (HC) and two light chains (LC) interconnected by disulfide bonds. The amino terminal portion of each chain includes a variable region of about 100 to about 110 amino acids primarily responsible for antigen recognition via the complementarity determining regions (CDRs) contained therein. The carboxy-terminal portion of each chain defines a constant region primarily responsible for effector function.

As used herein, the term “light chain variable region” or “LCVR” refers to a portion of a light chain of an antibody molecule that includes the amino acid sequences of CDRs and framework regions (FRs).

As used herein, the term “heavy chain variable region” “HCVR” refers to a portion of a heavy chain of an antibody molecule that includes the amino acid sequences of CDRs and FRs.

The antibodies described herein may readily be produced in mammalian cells, non-limiting examples of which includes CHO, NSO, HEK293 or COS cells. The host cells are cultured using techniques well known in the art. In this regard, an appropriate host cell can be either transiently or stably transfected with an expression system for secreting antibodies using an optimal predetermined HC:LC vector ratio or a single vector system encoding both HC (heavy chain) and LC (light chain). The vectors containing the polynucleotide sequences of interest (e.g., the polynucleotides encoding the polypeptides of the antibody and expression control sequences) can be transferred into the host cell by well-known methods, which may vary depending on the type of cellular host. Clarified media, into which the antibody has been secreted, may be purified using any of many commonly-used techniques. Various methods of protein purification may be employed and such methods are known in the art and described, for example, in Deutscher, Methods in Enzymology 182: 83-89 (1990) and Scopes, Protein Purification: Principles and Practice, 3rd Edition, Springer, NY (1994). In some examples, the medium may be conveniently applied to a column that has been equilibrated with a compatible buffer. The column may be washed to remove nonspecific binding components. The bound antibody may be eluted, for example, by pH gradient. Antibody fractions may be detected, such as by UV absorbance or SDS-PAGE, and then may be pooled. Further purification is optional, depending on the intended use. The antibody may be concentrated and/or sterile filtered using common techniques. Soluble aggregate and multimers may be effectively removed by common techniques, including size exclusion, hydrophobic interaction, ion exchange, multimodal, or hydroxyapatite chromatography. The purity of the antibody after these chromatography steps is typically greater than 95%. The product may be immediately frozen at −70° C. or may be lyophilized.

The present disclosure provides a method of treating metastatic colorectal cancer in a patient in need of therapy, comprising measuring an amount of human VEGF-D (SEQ ID NO: 6) in a biological sample from the patient and subsequently administering a therapeutically effective amount of an anti-human VEGFR-2 (SEQ ID NO: 5) antibody to the patient.

The present disclosure provides a method of treating metastatic colorectal cancer in a patient in need of therapy, comprising measuring an amount of human VEGF-D (SEQ ID NO: 6) in a biological sample from the patient and subsequently administering a therapeutically effective amount of an anti-human VEGFR-2 (SEQ ID NO: 5) antibody to the patient; wherein the anti-human VEGFR-2 antibody comprises a heavy chain variable region having the amino acid sequence of SEQ ID NO: 2 and a light chain variable region having the amino acid sequence of SEQ ID NO: 1. The present disclosure provides a method of treating metastatic colorectal cancer in a patient in need of therapy, comprising measuring an amount of human VEGF-D (SEQ ID NO: 6) in a biological sample from the patient and subsequently administering a therapeutically effective amount of an anti-human VEGFR-2 (SEQ ID NO: 5) antibody to the patient; wherein the anti-human VEGFR-2 antibody further comprises a heavy chain having the amino acid sequence of SEQ ID NO: 4 and a light chain having the amino acid sequence of SEQ ID NO: 3. The present disclosure provides a method of treating metastatic colorectal cancer in a patient in need of therapy, comprising measuring an amount of human VEGF-D (SEQ ID NO: 6) in a biological sample from the patient and subsequently administering a therapeutically effective amount of an anti-human VEGFR-2 (SEQ ID NO: 5) antibody to the patient; wherein the anti-human VEGFR-2 antibody is ramucirumab.

The present disclosure provides a method of treating metastatic colorectal cancer in a patient in need of therapy, comprising measuring an amount of human VEGF-D (SEQ ID NO: 6) in a biological sample from the patient and subsequently administering a therapeutically effective amount of an anti-human VEGFR-2 (SEQ ID NO: 5) antibody to the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered if the level of VEGF-D in the biological sample is about 76 pg/ml or greater.

The present disclosure provides a method of treating metastatic colorectal cancer in a patient in need of therapy, comprising measuring an amount of human VEGF-D (SEQ ID NO: 6) in a biological sample from the patient and subsequently administering a therapeutically effective amount of an anti-human VEGFR-2 (SEQ ID NO: 5) antibody to the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered if the level of VEGF-D in the biological sample is from about 76 pg/ml to about 400 pg/ml.

The present disclosure provides a method of treating metastatic colorectal cancer in a patient in need of therapy, comprising measuring an amount of human VEGF-D (SEQ ID NO: 6) in a biological sample from the patient and subsequently administering a therapeutically effective amount of an anti-human VEGFR-2 (SEQ ID NO: 5) antibody to the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered if the level of VEGF-D in the biological sample is about 115 pg/ml or greater.

The present disclosure provides a method of treating metastatic colorectal cancer in a patient in need of therapy, comprising measuring an amount of human VEGF-D (SEQ ID NO: 6) in a biological sample from the patient and subsequently administering a therapeutically effective amount of an anti-human VEGFR-2 (SEQ ID NO: 5) antibody to the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the biological sample comprises serum derived from the patient or plasma derived from the patient; optionally wherein the biological sample further comprises heparin or ethylenediaminetetraacetic acid.

The present disclosure provides a method of treating metastatic colorectal cancer in a patient in need of therapy, comprising measuring an amount of human VEGF-D (SEQ ID NO: 6) in a biological sample from the patient and subsequently administering a therapeutically effective amount of an anti-human VEGFR-2 (SEQ ID NO: 5) antibody to the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered at a dose of 8 mg/kg every 2 weeks.

The present disclosure provides a method of treating metastatic colorectal cancer in a patient in need of therapy, comprising measuring an amount of human VEGF-D (SEQ ID NO: 6) in a biological sample from the patient and subsequently administering a therapeutically effective amount of an anti-human VEGFR-2 (SEQ ID NO: 5) antibody to the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay.

The present disclosure provides a method of treating metastatic colorectal cancer in a patient in need of therapy, comprising measuring an amount of human VEGF-D (SEQ ID NO: 6) in a biological sample from the patient and subsequently administering a therapeutically effective amount of an anti-human VEGFR-2 (SEQ ID NO: 5) antibody to the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D.

The present disclosure provides a method of treating metastatic colorectal cancer in a patient in need of therapy, comprising measuring an amount of human VEGF-D (SEQ ID NO: 6) in a biological sample from the patient and subsequently administering a therapeutically effective amount of an anti-human VEGFR-2 (SEQ ID NO: 5) antibody to the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D; wherein the immunoassay further comprises a capture antibody that is a mouse anti-human VEGF-D antibody and/or a detection antibody that is a mouse anti-human VEGF-D antibody.

The present disclosure provides a method of treating metastatic colorectal cancer in a patient in need of therapy, comprising measuring an amount of human VEGF-D (SEQ ID NO: 6) in a biological sample from the patient and subsequently administering a therapeutically effective amount of an anti-human VEGFR-2 (SEQ ID NO: 5) antibody to the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D; wherein the immunoassay further comprises a capture antibody that is a mouse anti-human VEGF-D antibody and a detection antibody that is a mouse anti-human VEGF-D antibody; wherein the capture antibody is labeled with biotin and the detection antibody is labeled with ruthenium or horseradish peroxide.

The present disclosure provides a method of treating metastatic colorectal cancer in a patient in need of therapy, comprising measuring an amount of human VEGF-D (SEQ ID NO: 6) in a biological sample from the patient and subsequently administering a therapeutically effective amount of an anti-human VEGFR-2 (SEQ ID NO: 5) antibody to the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the patient was previously treated with bevacizumab.

The present disclosure provides a method of treating metastatic colorectal cancer, comprising administering a therapeutically effective amount of an anti-human VEGFR-2 (SEQ ID NO: 5) antibody to a patient in need thereof, provided that the patient is selected for treatment after receiving a result from a test measuring the amount of human VEGF-D (SEQ ID NO: 6) in a biological sample from the patient.

The present disclosure provides a method of treating metastatic colorectal cancer, comprising administering a therapeutically effective amount of an anti-human VEGFR-2 (SEQ ID NO: 5) antibody to a patient in need thereof, provided that the patient is selected for treatment after receiving a result from a test measuring the amount of human VEGF-D (SEQ ID NO: 6) in a biological sample from the patient; wherein the anti-human VEGFR-2 antibody comprises a heavy chain variable region having the amino acid sequence of SEQ ID NO: 2 and a light chain variable region having the amino acid sequence of SEQ ID NO: 1. The present disclosure provides a method of treating metastatic colorectal cancer, comprising administering a therapeutically effective amount of an anti-human VEGFR-2 (SEQ ID NO: 5) antibody to a patient in need thereof, provided that the patient is selected for treatment after receiving a result from a test measuring the amount of human VEGF-D (SEQ ID NO: 6) in a biological sample from the patient; wherein the anti-human VEGFR-2 antibody further comprises a heavy chain having the amino acid sequence of SEQ ID NO: 4 and a light chain having the amino acid sequence of SEQ ID NO: 3. The present disclosure provides a method of treating metastatic colorectal cancer, comprising administering a therapeutically effective amount of an anti-human VEGFR-2 (SEQ ID NO: 5) antibody to a patient in need thereof, provided that the patient is selected for treatment after receiving a result from a test measuring the amount of human VEGF-D (SEQ ID NO: 6) in a biological sample from the patient; wherein the anti-human VEGFR-2 antibody is ramucirumab.

The present disclosure provides a method of treating metastatic colorectal cancer, comprising administering a therapeutically effective amount of an anti-human VEGFR-2 (SEQ ID NO: 5) antibody to a patient in need thereof, provided that the patient is selected for treatment after receiving a result from a test measuring the amount of human VEGF-D (SEQ ID NO: 6) in a biological sample from the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered if the level of VEGF-D in the biological sample is about 76 pg/ml or greater.

The present disclosure provides a method of treating metastatic colorectal cancer, comprising administering a therapeutically effective amount of an anti-human VEGFR-2 (SEQ ID NO: 5) antibody to a patient in need thereof, provided that the patient is selected for treatment after receiving a result from a test measuring the amount of human VEGF-D (SEQ ID NO: 6) in a biological sample from the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered if the level of VEGF-D in the biological sample is from about 76 pg/ml to about 400 pg/ml.

The present disclosure provides a method of treating metastatic colorectal cancer, comprising administering a therapeutically effective amount of an anti-human VEGFR-2 (SEQ ID NO: 5) antibody to a patient in need thereof, provided that the patient is selected for treatment after receiving a result from a test measuring the amount of human VEGF-D (SEQ ID NO: 6) in a biological sample from the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered if the level of VEGF-D in the biological sample is about 115 pg/ml or greater.

The present disclosure provides a method of treating metastatic colorectal cancer, comprising administering a therapeutically effective amount of an anti-human VEGFR-2 (SEQ ID NO: 5) antibody to a patient in need thereof, provided that the patient is selected for treatment after receiving a result from a test measuring the amount of human VEGF-D (SEQ ID NO: 6) in a biological sample from the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the biological sample comprises serum derived from the patient or plasma derived from the patient; optionally wherein the biological sample further comprises heparin or ethylenediaminetetraacetic acid.

The present disclosure provides a method of treating metastatic colorectal cancer, comprising administering a therapeutically effective amount of an anti-human VEGFR-2 (SEQ ID NO: 5) antibody to a patient in need thereof, provided that the patient is selected for treatment after receiving a result from a test measuring the amount of human VEGF-D (SEQ ID NO: 6) in a biological sample from the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered at a dose of 8 mg/kg every 2 weeks.

The present disclosure provides a method of treating metastatic colorectal cancer, comprising administering a therapeutically effective amount of an anti-human VEGFR-2 (SEQ ID NO: 5) antibody to a patient in need thereof, provided that the patient is selected for treatment after receiving a result from a test measuring the amount of human VEGF-D (SEQ ID NO: 6) in a biological sample from the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay.

The present disclosure provides a method of treating metastatic colorectal cancer, comprising administering a therapeutically effective amount of an anti-human VEGFR-2 (SEQ ID NO: 5) antibody to a patient in need thereof, provided that the patient is selected for treatment after receiving a result from a test measuring the amount of human VEGF-D (SEQ ID NO: 6) in a biological sample from the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D.

The present disclosure provides a method of treating metastatic colorectal cancer, comprising administering a therapeutically effective amount of an anti-human VEGFR-2 (SEQ ID NO: 5) antibody to a patient in need thereof, provided that the patient is selected for treatment after receiving a result from a test measuring the amount of human VEGF-D (SEQ ID NO: 6) in a biological sample from the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D; wherein the immunoassay further comprises a capture antibody that is a mouse anti-human VEGF-D antibody and/or a detection antibody that is a mouse anti-human VEGF-D antibody.

The present disclosure provides a method of treating metastatic colorectal cancer, comprising administering a therapeutically effective amount of an anti-human VEGFR-2 (SEQ ID NO: 5) antibody to a patient in need thereof, provided that the patient is selected for treatment after receiving a result from a test measuring the amount of human VEGF-D (SEQ ID NO: 6) in a biological sample from the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D; wherein the immunoassay further comprises a capture antibody that is a mouse anti-human VEGF-D antibody and a detection antibody that is a mouse anti-human VEGF-D antibody; wherein the capture antibody is labeled with biotin and the detection antibody is labeled with ruthenium or horseradish peroxide.

The present disclosure provides a method of treating metastatic colorectal cancer, comprising administering a therapeutically effective amount of an anti-human VEGFR-2 (SEQ ID NO: 5) antibody to a patient in need thereof, provided that the patient is selected for treatment after receiving a result from a test measuring the amount of human VEGF-D (SEQ ID NO: 6) in a biological sample from the patient; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the patient was previously treated with bevacizumab.

The present disclosure provides a method of identifying a patient with metastatic colorectal cancer for treatment with an anti-human VEGFR-2 (SEQ ID NO: 5) antibody, comprising testing a biological sample from the patient for the presence of human VEGF-D (SEQ ID NO: 6); wherein the patient is eligible for treatment with the anti-human VEGFR-2 antibody if human VEGF-D is present in the biological sample.

The present disclosure provides a method of identifying a patient with metastatic colorectal cancer for treatment with an anti-human VEGFR-2 (SEQ ID NO: 5) antibody, comprising testing a biological sample from the patient for the presence of human VEGF-D (SEQ ID NO: 6); wherein the patient is eligible for treatment with the anti-human VEGFR-2 antibody if human VEGF-D is present in the biological sample; wherein the anti-human VEGFR-2 antibody comprises a heavy chain variable region having the amino acid sequence of SEQ ID NO: 2 and a light chain variable region having the amino acid sequence of SEQ ID NO: 1. The present disclosure provides a method of identifying a patient with metastatic colorectal cancer for treatment with an anti-human VEGFR-2 (SEQ ID NO: 5) antibody, comprising testing a biological sample from the patient for the presence of human VEGF-D (SEQ ID NO: 6); wherein the patient is eligible for treatment with the anti-human VEGFR-2 antibody if human VEGF-D is present in the biological sample; wherein the anti-human VEGFR-2 antibody further comprises a heavy chain having the amino acid sequence of SEQ ID NO: 4 and a light chain having the amino acid sequence of SEQ ID NO: 3. The present disclosure provides a method of identifying a patient with metastatic colorectal cancer for treatment with an anti-human VEGFR-2 (SEQ ID NO: 5) antibody, comprising testing a biological sample from the patient for the presence of human VEGF-D (SEQ ID NO: 6); wherein the patient is eligible for treatment with the anti-human VEGFR-2 antibody if human VEGF-D is present in the biological sample; wherein the anti-human VEGFR-2 antibody is ramucirumab.

The present disclosure provides a method of identifying a patient with metastatic colorectal cancer for treatment with an anti-human VEGFR-2 (SEQ ID NO: 5) antibody, comprising testing a biological sample from the patient for the presence of human VEGF-D (SEQ ID NO: 6); wherein the patient is eligible for treatment with the anti-human VEGFR-2 antibody if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered if the level of VEGF-D in the biological sample is about 76 pg/ml or greater.

The present disclosure provides a method of identifying a patient with metastatic colorectal cancer for treatment with an anti-human VEGFR-2 (SEQ ID NO: 5) antibody, comprising testing a biological sample from the patient for the presence of human VEGF-D (SEQ ID NO: 6); wherein the patient is eligible for treatment with the anti-human VEGFR-2 antibody if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered if the level of VEGF-D in the biological sample is from about 76 pg/ml to about 400 pg/ml.

The present disclosure provides a method of identifying a patient with metastatic colorectal cancer for treatment with an anti-human VEGFR-2 (SEQ ID NO: 5) antibody, comprising testing a biological sample from the patient for the presence of human VEGF-D (SEQ ID NO: 6); wherein the patient is eligible for treatment with the anti-human VEGFR-2 antibody if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered if the level of VEGF-D in the biological sample is about 115 pg/ml or greater.

The present disclosure provides a method of identifying a patient with metastatic colorectal cancer for treatment with an anti-human VEGFR-2 (SEQ ID NO: 5) antibody, comprising testing a biological sample from the patient for the presence of human VEGF-D (SEQ ID NO: 6); wherein the patient is eligible for treatment with the anti-human VEGFR-2 antibody if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the biological sample comprises serum derived from the patient or plasma derived from the patient; optionally wherein the biological sample further comprises heparin or ethylenediaminetetraacetic acid.

The present disclosure provides a method of identifying a patient with metastatic colorectal cancer for treatment with an anti-human VEGFR-2 (SEQ ID NO: 5) antibody, comprising testing a biological sample from the patient for the presence of human VEGF-D (SEQ ID NO: 6); wherein the patient is eligible for treatment with the anti-human VEGFR-2 antibody if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered at a dose of 8 mg/kg every 2 weeks.

The present disclosure provides a method of identifying a patient with metastatic colorectal cancer for treatment with an anti-human VEGFR-2 (SEQ ID NO: 5) antibody, comprising testing a biological sample from the patient for the presence of human VEGF-D (SEQ ID NO: 6); wherein the patient is eligible for treatment with the anti-human VEGFR-2 antibody if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay.

The present disclosure provides a method of identifying a patient with metastatic colorectal cancer for treatment with an anti-human VEGFR-2 (SEQ ID NO: 5) antibody, comprising testing a biological sample from the patient for the presence of human VEGF-D (SEQ ID NO: 6); wherein the patient is eligible for treatment with the anti-human VEGFR-2 antibody if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D.

The present disclosure provides a method of identifying a patient with metastatic colorectal cancer for treatment with an anti-human VEGFR-2 (SEQ ID NO: 5) antibody, comprising testing a biological sample from the patient for the presence of human VEGF-D (SEQ ID NO: 6); wherein the patient is eligible for treatment with the anti-human VEGFR-2 antibody if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D; wherein the immunoassay further comprises a capture antibody that is a mouse anti-human VEGF-D antibody and/or a detection antibody that is a mouse anti-human VEGF-D antibody.

The present disclosure provides a method of identifying a patient with metastatic colorectal cancer for treatment with an anti-human VEGFR-2 (SEQ ID NO: 5) antibody, comprising testing a biological sample from the patient for the presence of human VEGF-D (SEQ ID NO: 6); wherein the patient is eligible for treatment with the anti-human VEGFR-2 antibody if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D; wherein the immunoassay further comprises a capture antibody that is a mouse anti-human VEGF-D antibody and a detection antibody that is a mouse anti-human VEGF-D antibody; wherein the capture antibody is labeled with biotin and the detection antibody is labeled with ruthenium or horseradish peroxide.

The present disclosure provides a method of identifying a patient with metastatic colorectal cancer for treatment with an anti-human VEGFR-2 (SEQ ID NO: 5) antibody, comprising testing a biological sample from the patient for the presence of human VEGF-D (SEQ ID NO: 6); wherein the patient is eligible for treatment with the anti-human VEGFR-2 antibody if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the patient was previously treated with bevacizumab.

The present disclosure provides a therapeutic regimen for treating metastatic colorectal cancer comprising: a) selecting a patient having metastatic colorectal cancer and whose levels of VEGF-D (SEQ ID NO: 6) in a biological sample is about 76 pg/ml or greater and b) administering to the patient a therapeutically effective amount of an anti-human VEGFR-2 (SEQ ID NO: 5) antibody.

The present disclosure provides a therapeutic regimen for treating metastatic colorectal cancer comprising: a) selecting a patient having metastatic colorectal cancer and whose levels of VEGF-D (SEQ ID NO: 6) in a biological sample is about 76 pg/ml or greater and b) administering to the patient a therapeutically effective amount of an anti-human VEGFR-2 (SEQ ID NO: 5) antibody; wherein the anti-human VEGFR-2 antibody comprises a heavy chain variable region having the amino acid sequence of SEQ ID NO: 2 and a light chain variable region having the amino acid sequence of SEQ ID NO: 1. The present disclosure provides a therapeutic regimen for treating metastatic colorectal cancer comprising: a) selecting a patient having metastatic colorectal cancer and whose levels of VEGF-D (SEQ ID NO: 6) in a biological sample is about 76 pg/ml or greater and b) administering to the patient a therapeutically effective amount of an anti-human VEGFR-2 (SEQ ID NO: 5) antibody; wherein the anti-human VEGFR-2 antibody further comprises a heavy chain having the amino acid sequence of SEQ ID NO: 4 and a light chain having the amino acid sequence of SEQ ID NO: 3. The present disclosure provides a therapeutic regimen for treating metastatic colorectal cancer comprising: a) selecting a patient having metastatic colorectal cancer and whose levels of VEGF-D (SEQ ID NO: 6) in a biological sample is about 76 pg/ml or greater and b) administering to the patient a therapeutically effective amount of an anti-human VEGFR-2 (SEQ ID NO: 5) antibody; wherein the anti-human VEGFR-2 antibody is ramucirumab.

The present disclosure provides a therapeutic regimen for treating metastatic colorectal cancer comprising: a) selecting a patient having metastatic colorectal cancer and whose levels of VEGF-D (SEQ ID NO: 6) in a biological sample is about 76 pg/ml or greater and b) administering to the patient a therapeutically effective amount of an anti-human VEGFR-2 (SEQ ID NO: 5) antibody; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered if the level of VEGF-D in the biological sample is about 76 pg/ml or greater.

The present disclosure provides a therapeutic regimen for treating metastatic colorectal cancer comprising: a) selecting a patient having metastatic colorectal cancer and whose levels of VEGF-D (SEQ ID NO: 6) in a biological sample is about 76 pg/ml or greater and b) administering to the patient a therapeutically effective amount of an anti-human VEGFR-2 (SEQ ID NO: 5) antibody; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered if the level of VEGF-D in the biological sample is from about 76 pg/ml to about 400 pg/ml.

The present disclosure provides a therapeutic regimen for treating metastatic colorectal cancer comprising: a) selecting a patient having metastatic colorectal cancer and whose levels of VEGF-D (SEQ ID NO: 6) in a biological sample is about 76 pg/ml or greater and b) administering to the patient a therapeutically effective amount of an anti-human VEGFR-2 (SEQ ID NO: 5) antibody; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered if the level of VEGF-D in the biological sample is about 115 pg/ml or greater.

The present disclosure provides a therapeutic regimen for treating metastatic colorectal cancer comprising: a) selecting a patient having metastatic colorectal cancer and whose levels of VEGF-D (SEQ ID NO: 6) in a biological sample is about 76 pg/ml or greater and b) administering to the patient a therapeutically effective amount of an anti-human VEGFR-2 (SEQ ID NO: 5) antibody; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the biological sample comprises serum derived from the patient or plasma derived from the patient; optionally wherein the biological sample further comprises heparin or ethylenediaminetetraacetic acid.

The present disclosure provides a therapeutic regimen for treating metastatic colorectal cancer comprising: a) selecting a patient having metastatic colorectal cancer and whose levels of VEGF-D (SEQ ID NO: 6) in a biological sample is about 76 pg/ml or greater and b) administering to the patient a therapeutically effective amount of an anti-human VEGFR-2 (SEQ ID NO: 5) antibody; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered at a dose of 8 mg/kg every 2 weeks.

The present disclosure provides a therapeutic regimen for treating metastatic colorectal cancer comprising: a) selecting a patient having metastatic colorectal cancer and whose levels of VEGF-D (SEQ ID NO: 6) in a biological sample is about 76 pg/ml or greater and b) administering to the patient a therapeutically effective amount of an anti-human VEGFR-2 (SEQ ID NO: 5) antibody; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay.

The present disclosure provides a therapeutic regimen for treating metastatic colorectal cancer comprising: a) selecting a patient having metastatic colorectal cancer and whose levels of VEGF-D (SEQ ID NO: 6) in a biological sample is about 76 pg/ml or greater and b) administering to the patient a therapeutically effective amount of an anti-human VEGFR-2 (SEQ ID NO: 5) antibody; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D.

The present disclosure provides a therapeutic regimen for treating metastatic colorectal cancer comprising: a) selecting a patient having metastatic colorectal cancer and whose levels of VEGF-D (SEQ ID NO: 6) in a biological sample is about 76 pg/ml or greater and b) administering to the patient a therapeutically effective amount of an anti-human VEGFR-2 (SEQ ID NO: 5) antibody; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D; wherein the immunoassay further comprises a capture antibody that is a mouse anti-human VEGF-D antibody and/or a detection antibody that is a mouse anti-human VEGF-D antibody.

The present disclosure provides a therapeutic regimen for treating metastatic colorectal cancer comprising: a) selecting a patient having metastatic colorectal cancer and whose levels of VEGF-D (SEQ ID NO: 6) in a biological sample is about 76 pg/ml or greater and b) administering to the patient a therapeutically effective amount of an anti-human VEGFR-2 (SEQ ID NO: 5) antibody; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D; wherein the immunoassay further comprises a capture antibody that is a mouse anti-human VEGF-D antibody and a detection antibody that is a mouse anti-human VEGF-D antibody; wherein the capture antibody is labeled with biotin and the detection antibody is labeled with ruthenium or horseradish peroxide.

The present disclosure provides a therapeutic regimen for treating metastatic colorectal cancer comprising: a) selecting a patient having metastatic colorectal cancer and whose levels of VEGF-D (SEQ ID NO: 6) in a biological sample is about 76 pg/ml or greater and b) administering to the patient a therapeutically effective amount of an anti-human VEGFR-2 (SEQ ID NO: 5) antibody; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the patient was previously treated with bevacizumab.

The present disclosure provides an anti-human VEGFR-2 (SEQ ID NO: 5) antibody for use in treating metastatic colorectal cancer, comprising: (a) performing an immunoassay for measuring the amount of human VEGF-D (SEQ ID NO: 6) in a biological sample from a patient; (b) determining the amount of human VEGF-D in the biological sample; and (c) administering a therapeutically effective amount of the anti-human VEGFR-2 antibody to the patient if human VEGF-D is present in the biological sample.

The present disclosure provides an anti-human VEGFR-2 (SEQ ID NO: 5) antibody for use in treating metastatic colorectal cancer, comprising: (a) performing an immunoassay for measuring the amount of human VEGF-D (SEQ ID NO: 6) in a biological sample from a patient; (b) determining the amount of human VEGF-D in the biological sample; and (c) administering a therapeutically effective amount of the anti-human VEGFR-2 antibody to the patient if human VEGF-D is present in the biological sample; wherein the anti-human VEGFR-2 antibody comprises a heavy chain variable region having the amino acid sequence of SEQ ID NO: 2 and a light chain variable region having the amino acid sequence of SEQ ID NO: 1. An anti-human VEGFR-2 (SEQ ID NO: 5) antibody for use in treating metastatic colorectal cancer, comprising: (a) performing an immunoassay for measuring the amount of human VEGF-D (SEQ ID NO: 6) in a biological sample from a patient; (b) determining the amount of human VEGF-D in the biological sample; and (c) administering a therapeutically effective amount of the anti-human VEGFR-2 antibody to the patient if human VEGF-D is present in the biological sample; wherein the anti-human VEGFR-2 antibody further comprises a heavy chain having the amino acid sequence of SEQ ID NO: 4 and a light chain having the amino acid sequence of SEQ ID NO: 3. An anti-human VEGFR-2 (SEQ ID NO: 5) antibody for use in treating metastatic colorectal cancer, comprising: (a) performing an immunoassay for measuring the amount of human VEGF-D (SEQ ID NO: 6) in a biological sample from a patient; (b) determining the amount of human VEGF-D in the biological sample; and (c) administering a therapeutically effective amount of the anti-human VEGFR-2 antibody to the patient if human VEGF-D is present in the biological sample; wherein the anti-human VEGFR-2 antibody is ramucirumab.

The present disclosure provides an anti-human VEGFR-2 (SEQ ID NO: 5) antibody for use in treating metastatic colorectal cancer, comprising: (a) performing an immunoassay for measuring the amount of human VEGF-D (SEQ ID NO: 6) in a biological sample from a patient; (b) determining the amount of human VEGF-D in the biological sample; and (c) administering a therapeutically effective amount of the anti-human VEGFR-2 antibody to the patient if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered if the level of VEGF-D in the biological sample is about 76 pg/ml or greater.

The present disclosure provides an anti-human VEGFR-2 (SEQ ID NO: 5) antibody for use in treating metastatic colorectal cancer, comprising: (a) performing an immunoassay for measuring the amount of human VEGF-D (SEQ ID NO: 6) in a biological sample from a patient; (b) determining the amount of human VEGF-D in the biological sample; and (c) administering a therapeutically effective amount of the anti-human VEGFR-2 antibody to the patient if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered if the level of VEGF-D in the biological sample is from about 76 pg/ml to about 400 pg/ml.

The present disclosure provides an anti-human VEGFR-2 (SEQ ID NO: 5) antibody for use in treating metastatic colorectal cancer, comprising: (a) performing an immunoassay for measuring the amount of human VEGF-D (SEQ ID NO: 6) in a biological sample from a patient; (b) determining the amount of human VEGF-D in the biological sample; and (c) administering a therapeutically effective amount of the anti-human VEGFR-2 antibody to the patient if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered if the level of VEGF-D in the biological sample is about 115 pg/ml or greater.

The present disclosure provides an anti-human VEGFR-2 (SEQ ID NO: 5) antibody for use in treating metastatic colorectal cancer, comprising: (a) performing an immunoassay for measuring the amount of human VEGF-D (SEQ ID NO: 6) in a biological sample from a patient; (b) determining the amount of human VEGF-D in the biological sample; and (c) administering a therapeutically effective amount of the anti-human VEGFR-2 antibody to the patient if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the biological sample comprises serum derived from the patient or plasma derived from the patient; optionally wherein the biological sample further comprises heparin or ethylenediaminetetraacetic acid.

The present disclosure provides an anti-human VEGFR-2 (SEQ ID NO: 5) antibody for use in treating metastatic colorectal cancer, comprising: (a) performing an immunoassay for measuring the amount of human VEGF-D (SEQ ID NO: 6) in a biological sample from a patient; (b) determining the amount of human VEGF-D in the biological sample; and (c) administering a therapeutically effective amount of the anti-human VEGFR-2 antibody to the patient if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered at a dose of 8 mg/kg every 2 weeks.

The present disclosure provides an anti-human VEGFR-2 (SEQ ID NO: 5) antibody for use in treating metastatic colorectal cancer, comprising: (a) performing an immunoassay for measuring the amount of human VEGF-D (SEQ ID NO: 6) in a biological sample from a patient; (b) determining the amount of human VEGF-D in the biological sample; and (c) administering a therapeutically effective amount of the anti-human VEGFR-2 antibody to the patient if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay.

The present disclosure provides an anti-human VEGFR-2 (SEQ ID NO: 5) antibody for use in treating metastatic colorectal cancer, comprising: (a) performing an immunoassay for measuring the amount of human VEGF-D (SEQ ID NO: 6) in a biological sample from a patient; (b) determining the amount of human VEGF-D in the biological sample; and (c) administering a therapeutically effective amount of the anti-human VEGFR-2 antibody to the patient if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D.

The present disclosure provides an anti-human VEGFR-2 (SEQ ID NO: 5) antibody for use in treating metastatic colorectal cancer, comprising: (a) performing an immunoassay for measuring the amount of human VEGF-D (SEQ ID NO: 6) in a biological sample from a patient; (b) determining the amount of human VEGF-D in the biological sample; and (c) administering a therapeutically effective amount of the anti-human VEGFR-2 antibody to the patient if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D; wherein the immunoassay further comprises a capture antibody that is a mouse anti-human VEGF-D antibody and/or a detection antibody that is a mouse anti-human VEGF-D antibody.

The present disclosure provides an anti-human VEGFR-2 (SEQ ID NO: 5) antibody for use in treating metastatic colorectal cancer, comprising: (a) performing an immunoassay for measuring the amount of human VEGF-D (SEQ ID NO: 6) in a biological sample from a patient; (b) determining the amount of human VEGF-D in the biological sample; and (c) administering a therapeutically effective amount of the anti-human VEGFR-2 antibody to the patient if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D; wherein the immunoassay further comprises a capture antibody that is a mouse anti-human VEGF-D antibody and a detection antibody that is a mouse anti-human VEGF-D antibody; wherein the capture antibody is labeled with biotin and the detection antibody is labeled with ruthenium or horseradish peroxide.

The present disclosure provides an anti-human VEGFR-2 (SEQ ID NO: 5) antibody for use in treating metastatic colorectal cancer, comprising: (a) performing an immunoassay for measuring the amount of human VEGF-D (SEQ ID NO: 6) in a biological sample from a patient; (b) determining the amount of human VEGF-D in the biological sample; and (c) administering a therapeutically effective amount of the anti-human VEGFR-2 antibody to the patient if human VEGF-D is present in the biological sample; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the patient was previously treated with bevacizumab.

The present disclosure provides a pharmaceutical composition comprising an anti-human VEGFR-2 (SEQ ID NO: 5) antibody with one or more pharmaceutically acceptable carriers, diluents, or excipients for use in the treatment of a patient having metastatic colorectal cancer, wherein a biological sample from the patient is measured for the level of human VEGF-D (SEQ ID NO: 6); wherein the anti-human VEGFR-2 antibody is administered if the level of VEGF-D in the biological sample is about 76 pg/ml, preferably from about 76 pg/ml to about 400 pg/ml, or more preferably about 115 pg/ml or greater.

The present disclosure provides a pharmaceutical composition comprising an anti-human VEGFR-2 (SEQ ID NO: 5) antibody with one or more pharmaceutically acceptable carriers, diluents, or excipients for use in the treatment of a patient having metastatic colorectal cancer, wherein a biological sample from the patient is measured for the level of human VEGF-D (SEQ ID NO: 6); wherein the anti-human VEGFR-2 antibody is administered if the level of VEGF-D in the biological sample is about 76 pg/ml, preferably from about 76 pg/ml to about 400 pg/ml, or more preferably about 115 pg/ml or greater; wherein the anti-human VEGFR-2 antibody comprises a heavy chain variable region having the amino acid sequence of SEQ ID NO: 2 and a light chain variable region having the amino acid sequence of SEQ ID NO: 1. The present disclosure provides a pharmaceutical composition comprising an anti-human VEGFR-2 (SEQ ID NO: 5) antibody with one or more pharmaceutically acceptable carriers, diluents, or excipients for use in the treatment of a patient having metastatic colorectal cancer, wherein a biological sample from the patient is measured for the level of human VEGF-D (SEQ ID NO: 6); wherein the anti-human VEGFR-2 antibody is administered if the level of VEGF-D in the biological sample is about 76 pg/ml, preferably from about 76 pg/ml to about 400 pg/ml, or more preferably about 115 pg/ml or greater; wherein the anti-human VEGFR-2 antibody further comprises a heavy chain having the amino acid sequence of SEQ ID NO: 4 and a light chain having the amino acid sequence of SEQ ID NO: 3. A pharmaceutical composition comprising an anti-human VEGFR-2 (SEQ ID NO: 5) antibody with one or more pharmaceutically acceptable carriers, diluents, or excipients for use in the treatment of a patient having metastatic colorectal cancer, wherein a biological sample from the patient is measured for the level of human VEGF-D (SEQ ID NO: 6); wherein the anti-human VEGFR-2 antibody is administered if the level of VEGF-D in the biological sample is about 76 pg/ml, preferably from about 76 pg/ml to about 400 pg/ml, or more preferably about 115 pg/ml or greater; wherein the anti-human VEGFR-2 antibody is ramucirumab.

The present disclosure provides a pharmaceutical composition comprising an anti-human VEGFR-2 (SEQ ID NO: 5) antibody with one or more pharmaceutically acceptable carriers, diluents, or excipients for use in the treatment of a patient having metastatic colorectal cancer, wherein a biological sample from the patient is measured for the level of human VEGF-D (SEQ ID NO: 6); wherein the anti-human VEGFR-2 antibody is administered if the level of VEGF-D in the biological sample is about 76 pg/ml, preferably from about 76 pg/ml to about 400 pg/ml, or more preferably about 115 pg/ml or greater; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the biological sample comprises serum derived from the patient or plasma derived from the patient; optionally wherein the biological sample further comprises heparin or ethylenediaminetetraacetic acid.

The present disclosure provides a pharmaceutical composition comprising an anti-human VEGFR-2 (SEQ ID NO: 5) antibody with one or more pharmaceutically acceptable carriers, diluents, or excipients for use in the treatment of a patient having metastatic colorectal cancer, wherein a biological sample from the patient is measured for the level of human VEGF-D (SEQ ID NO: 6); wherein the anti-human VEGFR-2 antibody is administered if the level of VEGF-D in the biological sample is about 76 pg/ml, preferably from about 76 pg/ml to about 400 pg/ml, or more preferably about 115 pg/ml or greater; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the anti-human VEGFR-2 antibody is administered at a dose of 8 mg/kg every 2 weeks.

The present disclosure provides a pharmaceutical composition comprising an anti-human VEGFR-2 (SEQ ID NO: 5) antibody with one or more pharmaceutically acceptable carriers, diluents, or excipients for use in the treatment of a patient having metastatic colorectal cancer, wherein a biological sample from the patient is measured for the level of human VEGF-D (SEQ ID NO: 6); wherein the anti-human VEGFR-2 antibody is administered if the level of VEGF-D in the biological sample is about 76 pg/ml, preferably from about 76 pg/ml to about 400 pg/ml, or more preferably about 115 pg/ml or greater; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay.

The present disclosure provides a pharmaceutical composition comprising an anti-human VEGFR-2 (SEQ ID NO: 5) antibody with one or more pharmaceutically acceptable carriers, diluents, or excipients for use in the treatment of a patient having metastatic colorectal cancer, wherein a biological sample from the patient is measured for the level of human VEGF-D (SEQ ID NO: 6); wherein the anti-human VEGFR-2 antibody is administered if the level of VEGF-D in the biological sample is about 76 pg/ml, preferably from about 76 pg/ml to about 400 pg/ml, or more preferably about 115 pg/ml or greater; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D.

The present disclosure provides a pharmaceutical composition comprising an anti-human VEGFR-2 (SEQ ID NO: 5) antibody with one or more pharmaceutically acceptable carriers, diluents, or excipients for use in the treatment of a patient having metastatic colorectal cancer, wherein a biological sample from the patient is measured for the level of human VEGF-D (SEQ ID NO: 6); wherein the anti-human VEGFR-2 antibody is administered if the level of VEGF-D in the biological sample is about 76 pg/ml, preferably from about 76 pg/ml to about 400 pg/ml, or more preferably about 115 pg/ml or greater; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D; wherein the immunoassay further comprises a capture antibody that is a mouse anti-human VEGF-D antibody and/or a detection antibody that is a mouse anti-human VEGF-D antibody.

The present disclosure provides a pharmaceutical composition comprising an anti-human VEGFR-2 (SEQ ID NO: 5) antibody with one or more pharmaceutically acceptable carriers, diluents, or excipients for use in the treatment of a patient having metastatic colorectal cancer, wherein a biological sample from the patient is measured for the level of human VEGF-D (SEQ ID NO: 6); wherein the anti-human VEGFR-2 antibody is administered if the level of VEGF-D in the biological sample is about 76 pg/ml, preferably from about 76 pg/ml to about 400 pg/ml, or more preferably about 115 pg/ml or greater; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the amount of human VEGF-D is measured using an immunoassay; optionally, wherein the immunoassay comprises a standard comprising human VEGF-D or recombinant human VEGF-D; wherein the immunoassay further comprises a capture antibody that is a mouse anti-human VEGF-D antibody and a detection antibody that is a mouse anti-human VEGF-D antibody; wherein the capture antibody is labeled with biotin and the detection antibody is labeled with ruthenium or horseradish peroxide.

The present disclosure provides a pharmaceutical composition comprising an anti-human VEGFR-2 (SEQ ID NO: 5) antibody with one or more pharmaceutically acceptable carriers, diluents, or excipients for use in the treatment of a patient having metastatic colorectal cancer, wherein a biological sample from the patient is measured for the level of human VEGF-D (SEQ ID NO: 6); wherein the anti-human VEGFR-2 antibody is administered if the level of VEGF-D in the biological sample is about 76 pg/ml, preferably from about 76 pg/ml to about 400 pg/ml, or more preferably about 115 pg/ml or greater; wherein the anti-human VEGFR-2 antibody: (a) comprises a heavy chain variable region having the amino acid sequence of SEQ ID NO: 2 and a light chain variable region having the amino acid sequence of SEQ ID NO: 1, (b) comprises a heavy chain having the amino acid sequence of SEQ ID NO: 4 and a light chain having the amino acid sequence of SEQ ID NO: 3, or (c) is ramucirumab; wherein the patient was previously treated with bevacizumab.

The present disclosure provides a method of treating metastatic colorectal cancer, comprising administering a therapeutically effective amount of an anti-human VEGFR-2 (SEQ ID NO: 5) antibody to a patient in need thereof, provided that the patient is selected for treatment after receiving a result from a measurement of the amount of human VEGF-D (SEQ ID NO: 6) in a biological sample from the patient; wherein the amount of human VEGF-D is measured using an immunoassay; wherein the immunoassay comprises a capture antibody comprising a heavy chain having the amino acid sequence of SEQ ID NO: 9 and a light chain having the amino acid sequence of SEQ ID NO: 10 and a detection antibody comprising a heavy chain having the amino acid sequence of SEQ ID NO: 11 and a light chain having the amino acid sequence of SEQ ID NO: 12.

The present disclosure provides a method of treating metastatic colorectal cancer, comprising administering a therapeutically effective amount of ramucirumab to a patient in need thereof, provided that the patient is selected for treatment if the level of human VEGF-D (SEQ ID NO: 6) in a biological sample derived from the patient is about 76 pg/ml or greater.

The present disclosure provides a pharmaceutical composition comprising an anti-human VEGFR-2 (SEQ ID NO: 5) antibody and one or more pharmaceutically acceptable carriers, diluents, or excipients for use in the treatment of a patient having metastatic colorectal cancer, wherein a biological sample from the patient is measured for the level of human VEGF-D (SEQ ID NO: 6); wherein the anti-human VEGFR-2 antibody is administered if the level of VEGF-D in the biological sample is about 76 pg/ml, preferably from about 76 pg/ml to about 400 pg/ml, or more preferably about 115 pg/ml or greater; wherein the amount of human VEGF-D is measured using an immunoassay comprising a capture antibody and a detection antibody; wherein the capture antibody comprises a heavy chain having the amino acid sequence of SEQ ID NO: 9 and a light chain having the amino acid sequence of SEQ ID NO: 10 and the detection antibody comprises a heavy chain having the amino acid sequence of SEQ ID NO: 11 and a light chain having the amino acid sequence of SEQ ID NO: 12.

A Randomized, Double-Blind, Multicenter Phase 3 Study of Irinotecan, Folinic Acid, and 5-Fluorouracil (FOLFIRI) Plus Ramucirumab or Placebo in Patients With Metastatic Colorectal Carcinoma Progressive During or Following First-Line Combination Therapy With Bevacizumab, Oxaliplatin, and a Fluoropyrimidine

Study Design:

The Study NCT01183780 is a Multicenter, Randomized, Double-Blind, Phase 3 Study of Irinotecan, Folinic Acid, and 5-Fluorouracil (FOLFIRI) Plus Ramucirumab (IMC-1121B) Drug Product or Placebo in Patients with Metastatic Colorectal Carcinoma Progressive During or Following First-Line Combination Therapy with Bevacizumab, Oxaliplatin, and a Fluoropyrimidine.

Approximately 1050 enrolled patients who meet all eligibility criteria are randomized into two Arms. Arm A patients receive FOLFIRI plus placebo (hereafter called Placebo arm) and Arm B patients receive FOLFIRI plus Ramucirumab (IMC-1121B) Drug Product (DP), (hereafter called Ramucirumab arm). Patients receive FOLFIRI plus placebo every 14 days or FOLFIRI plus ramucirumab DP every 14 days. At randomization, patients are stratified by geographic region (North America versus Europe versus all other regions), KRAS status (mutant versus wild-type), and time to disease progression after beginning first-line treatment (<6 months versus >6 months). Ramucirumab DP is a sterile, preservative-free solution for infusion and is formulated in an aqueous solution at a concentration of 10 mg/mL (500 mg/50-mL vial), administered as an intravenous (I.V.) infusion at a dose of 8 mg/kg every 2 weeks. The infusion is delivered in approximately 60 minutes. The infusion rate does not exceed 25 mg/minute. Placebo is a sterile, preservative-free solution for injection supplied in 50-mL vials. The volume of placebo to be administered will be calculated as if it were active product formulated at 10 mg/mL (administered at a dose of 8 mg/kg every 2 weeks). Commercial formulations of irinotecan, folinic acid (FA), and 5-fluorouracil (5-FU) are used and administered intravenously. Irinotecan at 180 mg/m² is administered intravenously over 90 minutes on Day 1 of each cycle, 1 hour after the end of the infusion of investigational drug product; followed by FA at 400 mg/m² administered intravenously over 120 minutes on Day 1 of each cycle (alternatively, FA may be administered [via separate infusion lines] concurrently with irinotecan); followed by 5-FU at 400 mg/m² bolus over 2 to 4 minutes administered intravenously immediately following completion of the FA infusion on Day 1 of each cycle; followed by 2400 mg/m² 5-FU which is administered intravenously over 46 to 48 hours (continuously) on Days 1 and 2 of each cycle.

Treatment is continued until disease progression, the development of unacceptable toxicity, noncompliance or withdrawal of consent by the patient, or investigator decision. Radiological evaluation of disease response is conducted approximately every 6 weeks (±3 days) through Week 36, then every 12 weeks (±3 days) thereafter, from first dose of study treatment until disease progression. Following discontinuation of study treatment, all patients are followed for survival at regularly scheduled intervals (every 3 months [±14 days]) as long as the patient remains alive or until study completion.

The primary efficacy analysis is performed in the intention-to-treat population, consisting of all randomized patients. The primary analysis compares the OS between the 2 treatment groups (with versus without ramucirumab DP) using the p-value from a log-rank test stratified by geographic region (North America versus Europe versus all other regions), KRAS status (mutant versus wild-type), and time to disease progression after beginning first-line treatment (<6 months versus ≥6 months). The estimation of survival curves for the 2 treatment groups are generated using the Kaplan-Meier methodology. A stratified Cox regression model to compare the treatments is performed to generate the HR and its 95% confidence limit. Overall survival is defined as the time from the date of randomization until the date of death from any cause. Progression-free survival is analyzed using the log-rank test, stratified by geographic region (North America versus Europe versus all other regions), KRAS status (mutant versus wild-type), and time to disease progression after beginning first-line treatment (<6 months versus ≥6 months). The stratified HR and its 95% confidence limit is estimated from a proportional hazards model (Cox model) with geographic region, KRAS status, and time to disease progression after beginning first-line treatment as stratification factors. The estimation of survival curves for the 2 treatment groups are generated using the Kaplan-Meier methodology. Progression-free survival is defined as the time from the date of randomization until the date of objectively determined progressive disease (according to RECIST v. 1.1) or death due to any cause, whichever is first. The ORR in each treatment group is compared using the Cochran-Mantel-Haenszel test adjusting for the stratification variables. Exact confidence bounds (confidence interval: 95%) are determined. The ORR (per RECIST v. 1.1) is defined as the proportion of patients with a best overall response of partial response or complete response.

Translational Research is conducted on plasma, whole blood, and archived tumor tissue samples collected from patients. Samples may be used for research on pathways associated with CRC, the mechanism of action of ramucirumab or FOLFIRI and/or angiogenesis, and may also be used for related research methods or validation of diagnostic tools or assay(s).

Biomarker Statistical Analysis for Clinical Study NCT01183780

Correlation studies between biomarkers with clinical outcomes are performed. An adaptive design (based on Freidlin and Simon 2005) is used to lend additional statistical robustness to the exploratory research. The study population is randomly and prospectively split into 2 sets in a ratio of 1:2, a marker exploratory set (ME) (where n is approximately 350 patients) and a marker confirmatory (MC) set (where n is approximately 700 patients), using stratification to balance the sets by the study stratification factors and treatment assignment.

Baseline assay results from the ME set are analyzed with clinical outcomes in an attempt to identify a binary classifier that predicts which patients are most likely to benefit from ramucirumab. Assay data from patients in the MC set are not available to the Sponsor during this analysis. If a potential classifier is identified, a prospective statistical analysis plan (SAP) for the MC set with respect to this classifier is created and locked by the Sponsor. This plan is such that the type I error rate within the MC set is controlled.

The primary objective of the analyses detailed in the SAP is to evaluate ramucirumab efficacy in the subgroups of patients from the RAISE trial (MC cohort and the ME+MC cohorts combined) with circulating vascular endothelial growth factor D (VEGF-D) levels that are greater than or equal to 115 pg/mL, and less than 115 pg/mL. In order to test the apparent relationship of VEGF-D levels with efficacy outcomes in the MC set of patients and in the full Translational Research (TR) population (ME+MC), a VEGF-D cut-off value is defined based on results from the ME set of patients. The information for both OS and PFS from statistical approaches including STEPP plots, diagnostic plots for dichotomization across the range of biomarker values, and differences in efficacy outcomes between the intent to treat (ITT) and ME set of patients is considered in a comprehensive empirical manner, resulting in the selection of 0.115 ng/mL (equivalent to 115 pg/mL) for the VEGF-D cutpoint. Plasma protein levels are reported as continuous variables. If the protein level in a sample is below the level that is quantifiable by the assay, then a BQL (below the limit of quantitation) result is reported for that sample.

Efficacy Analysis:

Association of VEGF-D Levels and Observed Treatment Effect with Ramucirumab:

In the ITT patient population, regardless of VEGF-D level, the median OS in the ramucirumab arm is 13.3 months, and the median OS in the placebo arm is 11.7 months. The stratified HR is 0.84 (95% CI: 0.73, 0.98; p=0.0219). The median PFS in the ramucirumab arm is 5.7 months, and the median PFS in the placebo arm is 4.5 months. The stratified HR is 0.79 (95% CI: 0.79, 0.90; p=0.0005).

ME and MC Combined Cohort Analysis:

In the combined ME and MC patient population (82% of the ITT population), the median OS in the ramucirumab arm is 13.3 months, and the median OS in the placebo arm is 12.0 months. The stratified HR is 0.89 (95% CI: 0.76, 1.04; p=0.1538). Furthermore, in the combined ME and MC patient population, the median PFS in the ramucirumab arm is 5.7 months, and the median PFS in the placebo arm is 4.8 months. The stratified HR is 0.89 (95% CI: 0.69, 0.92; p=0.0021).

ME and MC Cohort Analysis at a Pre-Speficied Cutpoint of 115 pg/mL:

Analyses using the pre-specified cutpoint of 115 pg/ml reveals that a poorer prognosis for patients was associated with elevated VEGF-D levels. When evaluating OS or PFS in the placebo arm, patients with VEGF-D levels greater than or equal to 115 pg/mL have a worse prognosis (median OS=11.53 months) than patients with levels less than 115 pg/mL (median OS=13.07 months). Similarly for PFS, patients with VEGF-D levels greater than or equal to 115 pg/mL have a worse prognosis (median PFS=4.2 months) than patients with levels less than 115 pg/mL (median PFS=5.61 months). These results indicate that VEGF-D is a prognostic factor for OS and PFS in mCRC patients.

The benefit of ramucirumab treatment is further assessed in the combined ME and MC patient population using the pre-specified cut-point of 115 pg/mL VEGF-D. The results shown in Table 2 and Table 3 below, show that ramucirumab has an increased benefit in patients with greater than or equal to 115 pg/mL VEGF-D levels in both OS and PFS. Results in the MC set alone show similar results, confirming the VEGF-D relationship independently of the ME analyses.

TABLE 3
PFS Analyses by VEGF-D 115 pg/mL cutpoint in combined ME and MC

| | VEGF-D < 115 pg/mL | | VEGF-D ≥ 115 pg/mL | |
|---|---|---|---|---|
| | Ramucirumab + | Placebo + | Ramucirumab + | Placebo + |
| | FOLFIRI | FOLFIRI | FOLFIRI | FOLFIRI |
| N | 176 | 172 | 270 | 266 |
| Median PFS, | 5.35 | 5.61 | 6.01 | 4.2 |
| months | | | | |
| 95% CI for | (4.20-5.78) | (5.28-6.93) | (5.55-6.96) | (4.07-4.50) |
| Median PFS | | | | |

| | | |
|---|---|---|
| Treatment HR | 1.16 | 0.62 |
| 95% CI for HR | (0.93-1.45) | (0.52-0.74) |
| p-value^{a} | 0.193 | <0.0001 |

^{a}P-value < 0.0001 from separate interaction analyses.

More granular analyses of the relationships of VEGF-D levels with OS and PFS are presented in Tables 4 and 5, respectively. These tables show the point estimates of treatment HRs (and associated 95% confidence intervals) for patients across the range of VEGF-D levels. Each HR point estimate is calculated from a subset analysis of a group of patients (N is approximately 200, as indicated) with VEGF-D levels whose median level is shown. A sliding window approach is used, in which the patients with the lowest VEGF-D levels are first analyzed and a HR calculated. The window is then slid toward slightly higher VEGF-D levels. Those patients with the lowest levels are removed from the group, and other patients with slightly higher VEGF-D levels are added to the group, and analysis is performed again. This is continued across the full range of VEGF-D levels observed in the trial. Table 4 below shows the Hazard Ratios Estimates for Overall Survival Analysis by Subpopulation from the TR Population with VEGF-D Data in combined ME+MC population (N=884)

(Ramucirumab+FOLFIRI versus Placebo+FOLFIRI).

Hazard Ratio greater than 1 indicates increasing hazard with Ramucirumab+FOLFIRI compared to Placebo+FOLFIRI.

ABBREVIATIONS: N=total number of patients, n=total number of patients within each subpopulation, CI=confidence interval, TR=translational research

TABLE 4

| | | | | Median | | |
|---|---|---|---|---|---|---|
| | | | | VEGF-D | Hazard Ratio | |
| Window | N | n1 | n2 | (ng/ml) | Estimate | 95% CI |
| 1 | 205 | 108 | 97 | 0.066 | 1.487 | (1.015, 2.179) |
| 2 | 201 | 111 | 90 | 0.076 | 1.279 | (0.889, 1.842) |
| 3 | 208 | 113 | 95 | 0.085 | 1.251 | (0.879, 1.783) |
| 4 | 205 | 108 | 97 | 0.096 | 1.141 | (0.799, 1.628) |
| 5 | 204 | 100 | 104 | 0.105 | 1.141 | (0.797, 1.635) |
| 6 | 201 | 94 | 107 | 0.112 | 1.046 | (0.729, 1.502) |
| 7 | 203 | 102 | 101 | 0.119 | 0.853 | (0.596, 1.221) |
| 8 | 202 | 110 | 92 | 0.1285 | 0.774 | (0.540, 1.111) |
| 9 | 204 | 113 | 91 | 0.141 | 0.714 | (0.502, 1.017) |
| 10 | 201 | 112 | 89 | 0.15 | 0.711 | (0.503, 1.005) |
| 11 | 200 | 107 | 93 | 0.161 | 0.727 | (0.513, 1.031) |
| 12 | 201 | 97 | 104 | 0.178 | 0.817 | (0.579, 1.153) |
| 13 | 200 | 91 | 109 | 0.193 | 0.811 | (0.577, 1.141) |
| 14 | 200 | 96 | 104 | 0.2155 | 0.715 | (0.504, 1.013) |
| 15 | 200 | 91 | 109 | 0.2325 | 0.757 | (0.538, 1.066) |
| 16 | 200 | 91 | 109 | 0.27 | 0.772 | (0.552, 1.080) |
| 17 | 183 | 89 | 94 | 0.301 | 0.654 | (0.453, 0.943) |

Table 5 below shows the Hazard Ratios Estimates for Progressive Free Survival Analysis by Subpopulation from the TR Population with VEGF-D Data in combined ME+MC population (N=884), (Ramucirumab+FOLFIRI versus Placebo+FOLFIRI). Hazard Ratio greater than 1 indicates increasing hazard with Ramucirumab+FOLFIRI compared to Placebo+FOLFIRI.

ABBREVIATIONS: N=total number of patients, n=total number of patients within each subpopulation, CI=confidence interval, TR=translational research

TABLE 5

| | | | | Median | | |
|---|---|---|---|---|---|---|
| | | | | VEGF-D | Hazard Ratio | |
| Window | N | n1 | n2 | (ng/ml) | Estimate | 95% CI |
| 1 | 205 | 108 | 97 | 0.066 | 1.258 | (0.918, 1.724) |
| 2 | 201 | 111 | 90 | 0.076 | 1.104 | (0.805, 1.514) |
| 3 | 208 | 113 | 95 | 0.085 | 1.053 | (0.775, 1.431) |
| 4 | 205 | 108 | 97 | 0.096 | 1.140 | (0.836, 1.553) |
| 5 | 204 | 100 | 104 | 0.105 | 0.979 | (0.712, 1.347) |
| 6 | 201 | 94 | 107 | 0.112 | 0.997 | (0.722, 1.376) |
| 7 | 203 | 102 | 101 | 0.119 | 0.939 | (0.681, 1.296) |
| 8 | 202 | 110 | 92 | 0.1285 | 0.721 | (0.521, 0.999) |
| 9 | 204 | 113 | 91 | 0.141 | 0.694 | (0.506, 0.952) |
| 10 | 201 | 112 | 89 | 0.15 | 0.665 | (0.482, 0.918) |
| 11 | 200 | 107 | 93 | 0.161 | 0.650 | (0.469, 0.901) |
| 12 | 201 | 97 | 104 | 0.178 | 0.710 | (0.519, 0.971) |
| 13 | 200 | 91 | 109 | 0.193 | 0.629 | (0.456, 0.867) |
| 14 | 200 | 96 | 104 | 0.2155 | 0.640 | (0.464, 0.884) |
| 15 | 200 | 91 | 109 | 0.2325 | 0.579 | (0.418, 0.802) |
| 16 | 200 | 91 | 109 | 0.27 | 0.586 | (0.427, 0.804) |
| 17 | 183 | 89 | 94 | 0.301 | 0.529 | (0.376, 0.746) |

As shown in Table 6 and Table 7 below, a VEGF-D level of 76 pg/ml may also be a suitable value that is useful to identify patients with greater benefit from ramucirumab treatment.

TABLE 6
OS Analyses by VEGF-D 76 pg/mL cutpoint in combined ME and MC

| | VEGF-D < 76 pg/mL | | VEGF-D < 76 pg/mL | |
|---|---|---|---|---|
| | Ramucirumab + | Placebo + | Ramucirumab + | |
| | FOLFIRI | FOLFIRI | FOLFIRI | Placebo + FOLFIRI |
| N | 72 | 74 | 374 | 364 |
| Median OS, | 13.96 | 17.64 | 13.14 | 11.63 |
| months | | | | |
| 95% CI for | (11.82-16.82) | (12.35-21.97) | (11.99-14.32) | (10.67-12.41) |
| Median OS | | | | |

| | | |
|---|---|---|
| Treatment | 1.68 | 0.84 |
| HR | | |
| 95% CI for | (1.09-2.59) | (0.71-0.99) |
| HR | | |
| p-value* | 0.0177 | 0.0430 |

P-value = 0.0195 from separate interaction analyses.

TABLE 7
PFS Analyses by VEGF-D 76 pg/mL cutpoint in combined ME and MC

| | VEGF-D < 76 pg/mL | | VEGF-D ≥ 76 pg/mL | |
|---|---|---|---|---|
| | Ramucirumab + | Placebo + | Ramucirumab + | Placebo + |
| | FOLFIRI | FOLFIRI | FOLFIRI | FOLFIRI |
| N | 72 | 74 | 374 | 364 |
| Median PFS, | 5.55 | 6.89 | 5.65 | 4.36 |
| months | | | | |
| 95% CI for Median | (4.33-7.16 | (4.59-8.31) | (5.45-6.30) | (4.17-5.45) |
| PFS | | | | |

| | | |
|---|---|---|
| Treatment HR | 1.27 | 0.75 |
| 95% CI for HR | (0.89-1.81) | (0.64-0.87) |
| p-value* | 0.1887 | 0.0002 |

P-value = 0.0351 from separate interaction analyses.

VEGF-D Assay

The purpose of this study is to assess the levels of human VEGF-D in samples derived from human blood samples utilizing the Meso Scale Discovery® Electrochemiluminescence (MSD-ECL) platform. The following MSD-ECL method is validated for the determination of VEGF-D in human serum, ethylenediaminetetraacetic acid (EDTA) plasma, and heparin plasma samples from clinical trials in a regulatory compliant manner. Validation of the method includes assessment of the minimum required dilution (MRD), standard curve accuracy and precision, intra- and inter assay precision, lower limit of quantification (LLOQ), dilutional linearity, normal human serum (NHS) sample reference range, spiked recovery, and short-term, long-term and freeze/thaw stability. Blood samples to determine human VEGF-D concentrations are collected at the specified time points and analyzed using the following immunoassay. The signal agent used with the detection antibody below may be altered as known in the art and in some examples an unlabeled capture antibody may be substituted; non-limiting examples of signal agents include the use of a colorimetric or a chemiluminescent conjugate instead of a ruthenium conjugate (e.g. horseradish peroxidase conjugate, phycoerythrin conjugate, biotin-conjugate, etc.). However, such modifications may affect the sensitivity of the assay, and as a result, the modified assay should be calibrated with the assay described below for best results.

In this assay, plates (streptavidin MSD ELISA plates, Cat. L15SA-1) are washed three times with 350 μL/well of wash buffer (1×TBST buffer), and then blocked with 200 μL/well blocking buffer (1×TBST buffer containing 1% bovine serum albumin). TBST is obtained from Boston Bioproducts Cat. IBB-181X (20×TBST). Plates are sealed and incubated for approximately 1 hour at room temperature (RT) on a Titramax™ 1000 plate shaker set at approximately 600 rpm. Blocked plates are washed three times with 350 μL/well of wash buffer. Wells are coated with 5 μg/mL biotin-labeled mouse anti-human VEGF-D antibody (Bio-anti-VEGF-D) (R&D Systems, Cat. MAB2861) in Coating Buffer (1×TBST Buffer+0.1% BSA) at 50 μL/well. Alternatively, IBA111 that has a heavy chain and a light chain having the amino acid sequence as shown in SEQ ID NO: 9 and SEQ ID NO: 10, respectively, may be used in place of MAB2861. Plates are sealed and incubated for approximately 1 hour at RT on a Titramax™ 1000 plate shaker set at approximately 600 rpm. During the bio-anti-VEGF-D incubation, all plasma samples are spun at approximately 14,000 rpm for 10 minutes and serum samples are spun if necessary.

Before the end of the incubation, a standard curve is prepared in a volume appropriate for 2 replicates for each point of the standard curve by diluting VEGF-D standard in dilution buffer (assay buffer+0.1% BSA+100 μg/mL heterophilic blocking reagent 1). Recombinant human VEGF-D protein is used for the standard (R&D Systems, 622-VD-025). The standard curve is prepared as a 12 step dilution curve starting at 300 ng/mL and diluting down 1 to 3 (1 part standard, 2 parts dilution buffer) for a total of 11 dilutions with the 12th dilution being a zero VEGF-D point. Controls are pre-diluted 5-fold in serum. Samples and pre-diluted controls are diluted 2-fold in dilution buffer. Sufficient volume is prepared for two 50 μL/well duplicates. Samples are vortexed prior to addition to plates. After the incubation is complete, the plates are washed three times with 350 μL/well of wash buffer. 50 μL of standards, diluted controls and diluted samples are added to duplicate wells of the blocked and bio-anti-VEGF-D coated plates, unless otherwise specified. Plates are sealed and incubated for approximately 2 hours at RT on a Titramax 1000 plate shaker set at approximately 600 rpm. Plates are washed three times with 350 μL/well of wash buffer. 50 μL of 0.5 μg/mL ruthenium-labeled mouse anti-human VEGF-D antibody (Ru-anti-VEGF-D) (R&D Systems, Cat. MAB286) in assay buffer+0.1% BSA are added to each well. Alternatively, MA112 that has a heavy chain and a light chain having the amino acid sequence as shown in SEQ ID NO: 11 and SEQ ID NO: 12, respectively, may be used in place of MAB286. Plates are sealed and incubated for approximately 1 hour at RT on a Titramax 1000 plate shaker set at approximately 600 rpm. Plates are washed three times with 350 μL/well of wash buffer. 2×MSD Read Buffer T (Meso Scale Diagnostics, Cat. R92TC-2) is prepared, and 150 μL is added to each well. Plates are shaken briefly and read on a SECTOR Imager 6000 (620 nm) within 5 minutes of read buffer addition.

SEQUENCE LISTING
SEQ ID NO: 1
(Anti-Human VEGFR-2 Antibody, LCVR)
(Artificial Sequence)
DIQMTQSPSSVSASIGDRVTITCRASQGIDNWLGWYQQKPGKAPKLLI
YDASNLDTGVPSRFSGSGSGTYFTLTISSLQAEDFAVYFCQQAKAFPP
TFGGGTKVDIK
SEQ ID NO: 2
(Anti-Human VEGFR-2 Antibody, HCVR)
(Artificial Sequence)
EVQLVQSGGGLVKPGGSLRLSCAASGFTFSSYSMNWVRQAPGKGLEWV
SSISSSSSYIYYADSVKGRFTISRDNAKNSLYLQMNSLRAEDTAVYYC
ARVTDAFDIWGQGTMVTVSS
SEQ ID NO: 3
(Anti-Human VEGFR-2 Antibody, LC)
(Artificial Sequence)
DIQMTQSPSSVSASIGDRVTITCRASQGIDNWLGWYQQKPGKAPKLLI
YDASNLDTGVPSRFSGSGSGTYFTLTISSLQAEDFAVYFCQQAKAFPP
TFGGGTKVDIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC
SEQ ID NO: 4
(Anti-Human VEGFR-2 Antibody, HC)
(Artificial Sequence)
EVQLVQSGGGLVKPGGSLRLSCAASGFTFSSYSMNWVRQAPGKGLEWV
SSISSSSSYIYYADSVKGRFTISRDNAKNSLYLQMNSLRAEDTAVYYC
ARVTDAFDIWGQGTMVTVSSASTKGPSVLPLAPSSKSTSGGTAALGCL
VKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVF
LFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPGK
SEQ ID NO: 5
(Human VEGFR-2) (Homo Sapiens)
MQSKVLLAVALWLCVETRAASVGLPSVSLDLPRLSIQKDILTIKANTT
LQITCRGQRDLDWLWPNNQSGSEQRVEVTECSDGLFCKTLTIPKVIGN
DTGAYKCFYRETDLASVIYVYVQDYRSPFIASVSDQHGVVYITENKNK
TVVIPCLGSISNLNVSLCARYPEKRFVPDGNRISWDSKKGFTIPSYMI
SYAGMVFCEAKINDESYQSIMYIVVVVGYRIYDVVLSPSHGIELSVGE
KLVLNCTARTELNVGIDFNWEYPSSKHQHKKLVNRDLKTQSGSEMKKF
LSTLTIDGVTRSDQGLYTCAASSGLMTKKNSTFVRVHEKPFVAFGSGM
ESLVEATVGERVRIPAKYLGYPPPEIKWYKNGIPLESNHTIKAGHVLT
IMEVSERDTGNYTVILTNPISKEKQSHVVSLVVYVPPQIGEKSLISPV
DSYQYGTTQTLTCTVYAIPPPHHIHWYWQLEEECANEPSQAVSVTNPY
PCEEWRSVEDFQGGNKIEVNKNQFALIEGKNKTVSTLVIQAANVSALY
KCEAVNKVGRGERVISFHVTRGPEITLQPDMQPTEQESVSLWCTADRS
TFENLTWYKLGPQPLPIHVGELPTPVCKNLDTLWKLNATMFSNSTNDI
LIMELKNASLQDQGDYVCLAQDRKTKKRHCVVRQLTVLERVAPTITGN
LENQTTSIGESIEVSCTASGNPPPQIMWFKDNETLVEDSGIVLKDGNR
NLTIRRVRKEDEGLYTCQACSVLGCAKVEAFFIIEGAQEKTNLEIIIL
VGTAVIAMFFWLLLVIILRTVKRANGGELKTGYLSIVMDPDELPLDEH
CERLPYDASKWEFPRDRLKLGKPLGRGAFGQVIEADAFGIDKTATCRT
VAVKMLKEGATHSEHRALMSELKILIHIGHHLNVVNLLGACTKPGGPL
MVIVEFCKFGNLSTYLRSKRNEFVPYKTKGARFRQGKDYVGAIPVDLK
RRLDSITSSQSSASSGFVEEKSLSDVEEEEAPEDLYKDFLTLEHLICY
SFQVAKGMEFLASRKCIHRDLAARNILLSEKNVVKICDFGLARDIYKD
PDYVRKGDARLPLKWMAPETIFDRVYTIQSDVWSFGVLLWEIFSLGAS
PYPGVKIDEEFCRRLKEGTRMRAPDYTTPEMYQTMLDCWHGEPSQRPT
FSELVEHLGNLLQANAQQDGKDYIVLPISETLSMEEDSGLSLPTSPVS
CMEEEEVCDPKFHYDNTAGISQYLQNSKRKSRPVSVKTFEDIPLEEPE
VKVIPDDNQTDSGMVLASEELKTLEDRTKLSPSFGGMVPSKSRESVAS
EGSNQTSGYQSGYHSDDTDTTVYSSEEAELLKLIEIGVQTGSTAQILQ
PDSGTTLSSPPV
SEQ ID NO: 6
(Human VEGF-D) (Homo Sapiens)
MYREWVVVNVFMMLYVQLVQGSSNEHGPVKRSSQSTLERSEQQIRAAS
SLEELLRITHSEDWKLWRCRLRLKSFTSMDSRSASHRSTRFAATFYDI
ETLKVIDEEWQRTQCSPRETCVEVASELGKSTNTFFKPPCVNVFRCGG
CCNEESLICMNTSTSYISKQLFEISVPLTSVPELVPVKVANHTGCKCL
PTAPRHPYSIIRRSIQIPEEDRCSHSKKLCPIDMLWDSNKCKCVLQEE
NPLAGTEDHSHLQEPALCGPHMMFDEDRCECVCKTPCPKDLIQHPKNC
SCFECKESLETCCQKHKLFHPDTCSCEDRCPFHTRPCASGKTACAKHC
RFPKEKRAAQGPHSRKNP
SEQ ID NO: 7
(Bevacizumab, HC) (Artificial Sequence)
EVQLVESGGGLVQPGGSLRLSCAASGYTFTNYGMNWVRQAPGKGLEWV
GWINTYTGEPTYAADFKRRFTFSLDTSKSTAYLQMNSLRAEDTAVYYC
AKYPHYYGSSHWYFDVWGQGTLVTVSSASTKGPSVFPLAPSSKSTSGG
TAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCS
VMHEALHNHYTQKSLSLSPGK
SEQ ID NO: 8
(Bevacizumab, LC) (Artificial Sequence)
DIQMTQSPSSLSASVGDRVTITCSASQDISNYLNWYQQKPGKAPKVLI
YFTSSLHSGVPSRFSGSGSGTDFTLTISSLQPEDFATYYCQQYSTVPW
TFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC
SEQ ID NO: 9
(IBA111, HC) (Artificial Sequence)
EVKLEESGGGLVQPGGSMKLSCVASGFTFSNYWMNWVRQSPEKGLEWV
AEITLKSNNYATHYAESVKGRFTISRDDSKSSVYLQMNNLRPEDTGIY
YCTRNDYDRAWFAYWGQGTLVTVSAAKTTAPSVYPLAPVCGDTTGSSV
TLGCLVKGYFPEPVTLTWNSGSLSSGVHTFPAVLQSDLYTLSSSVTVT
SSTWPSQSITCNVAHPASSTKVDKKIEPRGPTIKPCPPCKCPAPNLLG
GPSVFIFPPKIKDVLMISLSPIVTCVVVDVSEDDPDVQISWFVNNVEV
HTAQTQTHREDYNSTLRVVSALPIQHQDWMSGKEFKCKVNNKDLPAPI
ERTISKPKGSVRAPQVYVLPPPEEEMTKKQVTLTCMVTDFMPEDIYVE
WTNNGKTELNYKNTEPVLDSDGSYFMYSKLRVEKKNWVERNSYSCSVV
HEGLHNHHTTKSFSRTPGK
SEQ ID NO: 10
(IBA111, LC) (Artificial Sequence)
DIVMSQSPSSLAVSVGEKVTLSCKSSQSLLYSSNQKNYLAWYQQEPGQ
SPKLLIYWASTRESGVPDRFTGSGSGTDFTLTISSVKAEDLAVYYCQQ
YYNYPWTFGGGSKLEIKRADAAPTVSIFPPSSEQLTSGGASVVCFLNN
FYPKDINVKWKIDGSERQNGVLNSWTDQDSKDSTYSMSSTLTLTKDEY
ERHNSYTCEATHKTSTSPIVKSFNRNEC
SEQ ID NO: 11
(IBA111, HC) (Artificial Sequence)
EVQLQESGPSLVKPSQTLSLTCSVTGDSFTSDYWNWIRKFPGNRLEYM
GYISYRGVTYYNPSLKSRISITRDTSKNQYYLQLNSVTTEDTATYYCA
RWGTVPLDYWGQGTSVTVSSAKTTAPSVYPLAPVCGDTTGSSVTLGCL
VKGYFPEPVTLTWNSGSLSSGVHTFPAVLQSDLYTLSSSVTVTSSTWP
SQSITCNVAHPASSTKVDKKIEPRGPTIKPCPPCKCPAPNLLGGPSVF
IFPPKIKDVLMISLSPIVTCVVVDVSEDDPDVQISWFVNNVEVHTAQT
QTHREDYNSTLRVVSALPIQHQDWMSGKEFKCKVNNKDLPAPIERTIS
KPKGSVRAPQVYVLPPPEEEMTKKQVTLTCMVTDFMPEDIYVEWTNNG
KTELNYKNTEPVLDSDGSYFMYSKLRVEKKNWVERNSYSCSVVHEGLH
NHHTTKSFSRTPGK
SEQ ID NO: 12
(IBA112, LC) (Artificial Sequence)
DVLMTQTPLSLPVSLGDQASISCRSSQSIVHSNGNTYLEWYLQKPGQS
PKLLIYKVSNRFSGVPDRFSGSGSGTDFTLKISRVEAEDLGVYYCFQG
SHIPYTFGGGTKLEMKRADAAPTVSIFPPSSEQLTSGGASVVCFLNNF
YPKDINVKWKIDGSERQNGVLNSWTDQDSKDSTYSMSSTLTLTKDEYE
RHNSYTCEATHKTSTSPIVKSFNRNEC

1. (canceled)

2. A method of treating metastatic colorectal cancer,
comprising administering a therapeutically effective amount of an anti-human VEGFR-2 (SEQ ID NO: 5) antibody to a patient in need thereof, provided that the patient is selected for treatment after receiving a result from a measurement of the amount of human VEGF-D (SEQ ID NO: 6) in a biological sample from the patient; wherein the anti-human VEGFR-2 antibody comprises a heavy chain variable region having the amino acid sequence of SEQ ID NO: 2 and a light chain variable region having the amino acid sequence of SEQ ID NO: 1.

3. The method of claim 2, wherein the anti-human VEGFR-2 antibody is ramucirumab.

4. The method of claim 2, wherein the patient was previously treated with bevacizumab.

5. The method of claim 2, wherein the anti-human VEGFR-2 antibody is administered at a dose of about 8 mg/kg every 2 weeks.

6. (canceled)

7. The method of claim 2, wherein the anti-human VEGFR-2 antibody is administered if the level of human VEGF-D in the biological sample is from about 76 pg/ml to about 400 pg/ml.

8. (canceled)

9. The method of claim 2, wherein the biological sample comprises plasma or serum derived from the patient.

10. The method of claim 9, wherein the biological sample further comprises heparin.

11. The method of claim 9, wherein the biological sample further comprises ethylenediaminetetraacetic acid.

12. The method of claim 2, wherein the amount of human VEGF-D is measured using an immunoassay.

13. The method of claim 12, wherein the immunoassay comprises a standard that comprises human VEGF-D or recombinant human VEGF-D.

14. The method of claim 12, wherein the immunoassay comprises a capture antibody that is a mouse anti-human VEGF-D antibody.

15. The method of claim 14, wherein the capture antibody is labeled with biotin.

16. The method of claim 12, wherein the immunoassay comprises a detection antibody that is a mouse anti-human VEGF-D antibody.

17. The method of claim 16, wherein the detection antibody is labeled with ruthenium or horseradish peroxide.

18. The method of claim 14, wherein the capture antibody comprises a heavy chain having the amino acid sequence of SEQ ID NO: 9 and a light chain having the amino acid sequence of SEQ ID NO: 10.

19. The method of claim 16, wherein the detection antibody comprises a heavy chain having the amino acid sequence of SEQ ID NO: 11 and a light chain having the amino acid sequence of SEQ ID NO: 12.

20.-35. (canceled)
